(19) United States
(12) Patent Application Publication
(10) Pub. No.: US 2015/0224089 A1
(43) Pub. Date: Aug. 13, 2015

(54) COMBINATIONS OF IKKi/TBK1 INHIBITORS WITH BETA ADRENERGIC AGONISTS OR SYMPATHETIC NERVOUS SYSTEM ACTIVATORS

(71) Applicant: THE REGENTS OF THE UNIVERSITY OF MICHIGAN, Ann Arbor, MI (US)

(72) Inventor: Alan R. Saltiel, Ann Arbor, MI (US)

(21) Appl. No.: 14/615,478

(22) Filed: Feb. 6, 2015

Related U.S. Application Data
(60) Provisional application No. 61/937,302, filed on Feb. 7, 2014.

(51) Int. Cl. A61K 31/436; A61K 31/137

(52) U.S. Cl. CPC A61K 31/436; A61K 31/137

(57) ABSTRACT

Provided herein are methods of treating obesity and obesity-related conditions comprising the administration of combinations of IKKε/TBK1 inhibitors with beta adrenergic agonists or sympathetic nervous system activators, and pharmaceutical compositions comprising such combinations.

### CROSS REFERENCE TO RELATED APPLICATION

The present application claims priority to U.S. Provisional Patent Application Ser. No. 61/937,302, filed Feb. 7, 2014, the disclosure of which is herein incorporated by reference in its entirety.

### STATEMENT REGARDING GOVERNMENT FUNDING

This invention was made with government support under DK060591 awarded by the National Institutes of Health. The Government has certain rights in the invention.

### FIELD

Provided herein are methods of treating obesity and obesity-related conditions comprising the administration of combinations of IKKε/TBK1 inhibitors with beta adrenergic agonists or sympathetic nervous system activators, and pharmaceutical compositions comprising such combinations.

### BACKGROUND

Obesity generates a state of chronic, low-grade inflammation in liver and adipose tissue accompanied by macrophage infiltration and the local secretion of inflammatory cytokines and chemokines that attenuate insulin action, resulting in insulin resistance and the subsequent development of Type 2 diabetes (Wellen and Hotamisligil, 2005; Hotamisligil, 2006; Lumeng et al., 2007; Shoelson et al., 2007; herein incorporated by reference in their entireties). Numerous studies indicate a strong correlation between inflammation and insulin resistance across several populations (Hotamisligil, 2006; herein incorporated by reference in its entirety). Moreover, genetic ablation or pharmacological inhibition of inflammatory pathways can dissociate obesity from insulin resistance (Hotamisligil, 2006; Shoelson et al., 2007; herein incorporated by reference in their entireties).

The transcription factor NFκB and its inflammatory program play an important role in the development of insulin resistance in obese liver and adipose tissue (Yuan et al., 2001; Arkan et al., 2005; Wunderlich et al., 2008; Chiang et al., 2009; herein incorporated by reference in their entireties). NFκB is activated by the IκB kinase (IKK) family, which has four members: IKKα, IKKβ, IKKε, and TBK1. IKKα and IKKβ act with the scaffolding partner NEMO to activate NFκB (Hacker and Karin, 2006; herein incorporated by reference in its entirety). Although pharmacologic inhibition or genetic ablation of IKKβ defined a role for this kinase in insulin resistance (Yuan et al., 2001; Arkan et al., 2005; herein incorporated by reference in their entireties), the roles of the noncanonical kinases IKKε and TBK1 are not certain.

Obesity is a complex metabolic disorder that is caused by increased food intake and decreased expenditure of energy. Obesity also increases the risk of developing type 2 diabetes, heart disease, stroke, arthritis, and certain cancers. There is considerable evidence to suggest that adipose tissue becomes less sensitive to catecholamines such as adrenaline in states of obesity, and that this reduced sensitivity in turn reduces energy expenditure. However, the details of this process are not fully understood.

### SUMMARY

Provided herein are methods of treating obesity and obesity-related conditions comprising the administration of combinations of IKKε/TBK1 inhibitors with beta adrenergic agonists or sympathetic nervous system activators, and pharmaceutical compositions comprising such combinations.

In some embodiments, the present invention provides methods of treating a subject having a condition associated with obesity, insulin resistance, or hepatic steatosis, comprising: administering to a subject having a condition associated with obesity, insulin resistance, or hepatic steatosis: (i) an IKKε and/or TBK1 inhibitor, and (ii) a beta adrenergic agonist or sympathetic nervous system activator. In some embodiments, administering causes a reduction of body fat in the subject. In some embodiments, the subject has or is at risk of experiencing obesity, diabetes, or insulin resistance. In some embodiments, diabetes is type II diabetes. In some embodiments, the treatment results in increased glucose metabolism, reduction in body fat, lack of increase in body fat, increased insulin receptor signaling, decreased level of insulin receptor phosphorylation, reduction in or prevention of chronic inflammation in the liver, reduction in or prevention of chronic inflammation in adipose tissue, reduction in or prevention of hepatic steatosis, promotion of metabolic energy expenditure, reduction in circulating free fatty acids, or reduction in cholesterol. In some embodiments, the subject has hepatic steatosis (fatty liver disease). In some embodiments, the subject has steatohepatitis. In some embodiments, the subject is overweight or obese. In some embodiments, the subject is human.

In some embodiments, methods further comprises a step comprising testing the subject for a disease or condition selected from the group consisting of impaired insulin signaling, obesity, diabetes, insulin resistance, metabolic syndrome, hepatic steatosis, chronic liver inflammation, and chronic inflammation in adipose tissue. In some embodiments, method further comprises a step of assessing the effectiveness of treatment based upon said testing. In some embodiments, method further comprises adjusting the treatment based on said assessing. In some embodiments, adjusting the treatment comprises one or more of altering the dose of IKKε/TBK1 inhibitor, switching to a different IKKε/TBK1 inhibitor, altering the dose of beta adrenergic agonist or sympathetic nervous system activator, switching to a different beta adrenergic agonist or sympathetic nervous system activator, adding additional treatment.

In some embodiments, a method comprises administering a IKKε and/or TBK1 inhibitor and a beta adrenergic agonist or sympathetic nervous system activator that are co-formulated in a single pharmaceutical composition. In other embodiments, the IKKε and/or TBK1 inhibitor, and the beta adrenergic agonist or sympathetic nervous system activator are separate pharmaceutical compositions and are co-administered (e.g., within 1 hour, within 20 minutes, within 15 minutes, within 5 minutes, within 1 minute, simultaneously, etc.).

In some embodiments, the present invention provides pharmaceutical compositions comprising: (i) an IKKε and/or TBK1 inhibitor, and (ii) a beta adrenergic agonist or sympathetic nervous system activator. In some embodiments, the IKKε and/or TBK1 inhibitor comprises a small molecule. In some embodiments, the IKKε and/or TBK1 inhibitor comprises the structure of Formula I:

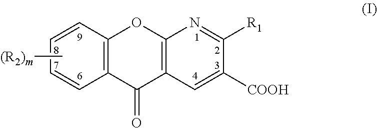

wherein R₁ is a hydrogen, alkyl, phenyl, carboxyl, hydroxyl, alkoxy, or amino group, which may be unsubstituted or substituted by one alkyl; m is 0, 1 or 2; and R₂ is alkyl, alkoxy, halogen, nitro, hydroxy, carboxyl, butadienylene (—CH═CH—CH═CH—), which forms a benzene ring with any adjacent carbon atoms or amino group, which may be unsubstituted or substituted by at least one alkyl, and their physiologically acceptable salts. In some embodiments, the IKKε and/or TBK1 inhibitor comprises amlexanox. In some embodiments, the beta adrenergic agonist or sympathetic nervous system activator comprises a small molecule. In some embodiments, the beta adrenergic agonist or sympathetic nervous system activator comprises a β2 adrenergic receptor agonist. In some embodiments, the small molecule beta adrenergic agonist or sympathetic nervous system activator is phentermine.

### BRIEF DESCRIPTION OF THE DRAWINGS

FIG. 1 shows IKKε and TBK1 overexpression decrease sensitivity to the β-adrenergic/cAMP pathway in 3T3-L1 adipocytes. (A) Fold increase in Ucp1 expression in 3T3-L1 adipocytes expressing empty vector, Flag-IKKε, or Flag-IKKε K38A following treatment with or without 10 μM ISO (black bars) or 10 μM CL-316,243 (CL, gray bars) for 4 hr. (B) Glycerol release from 3T3-L1 adipocytes expressing empty vector (white bars), Flag-IKKε (black bars), or Flag-IKKε K38A (gray bars) treated with or without 10 μM ISO or 10 μM CL. (C) Immunoblots of whole cell lysates from FIG. 1B. Results were replicated in triplicate. D.E. stands for dark exposure and L.E. stands for light exposure. (D) Immunoblots of whole cell lysates from 3T3-L1 adipocytes expressing empty vector or Flag-IKKε treated with or without 50 μM FSK for 15 min. (E) cAMP levels from 3T3-L1 adipocytes expressing empty vector, Flag-IKKε, or Flag-IKKε K38A treated with or without 10 μM ISO or 50 μM FSK for 15 min.

FIG. 2 shows Prolonged treatment with TNFα decreases the sensitivity of adipocytes to β-adrenergic stimulation in a manner dependent on the activity of IKKε and TBK1. (A) Glycerol release from 3T3-L1 adipocytes treated with or without different concentrations of TNFα as indicated for 24 hr followed by treatment with or without 10 μM ISO or 50 μM FSK. (B) cAMP levels from 3T3-L1 adipocytes treated with or without 100 ng/ml TNFα for 24 hr followed by treatment with or without 10 μM ISO or 50 μM FSK in the presence or absence of pretreatment of 50 μM Amlexanox (Am). (C) cAMP levels from 3T3-L1 adipocytes treated with or without 100 ng/ml TNFα for 24 hr followed by treatment with or without 50 μM FSK in the presence or absence of pretreatment of 1 μM CAY10576 (CAY). (D) Immunoblots of whole cell lysates from 3T3-L1 adipocytes treated with or without different concentrations of TNFα as same as FIG. 2A for 24 hr followed by treatment with or without 10 μM ISO or 50 μM FSK. Results were replicated in multiple experiments. ‘[’ indicates total HSL. ‘n.s.’ represents non-specific band. Arrow indicates CGI-58. (E) Immunoblots of whole cell lysates from 3T3-L1 adipocytes treated with or without 50 ng/ml TNFα or 100 μg/ml poly (I:C) for 24 hr followed by treatment with or without 10 μM ISO for 15 min in the presence or absence of pretreatment with increasing concentrations (0, 10, 50, and 200 μM) of amlexanox for 30 min.

FIG. 3 shows IKKε and TBK1 reduce cAMP levels through activation of PDE3B. (A) cAMP levels from 3T3-L1 adipocytes expressing empty vector, Flag-IKKε, or Flag-TBK1 treated with or without 50 μM FSK, 250 μM IBMX, or together for 15 min. (B) cAMP levels from 3T3-L1 adipocytes expressing empty vector, Flag-IKKε, or Flag-TBK1 treated with or without 10 μM ISO or 50 μM FSK together with or without 10 μM Zardaverine (Zarda) for 15 min. (C) 32P phospho-image of in vitro kinase reaction using either immunoprecipitated HA-PDE3B from HEK293T cells or 1 μg MBP (myelin basic protein) as a substrate with recombinant kinases as indicated. (D) Immunoblots of immunoprecipitation with anti-HA antibodies followed by treatment with or without CIP (top panel) and whole cell lysates (bottom panel) from Cos-1 cells co-expressing HA-PDE3B with Flag-IKKε/TBK1 or Flag-IKKε/TBK1 K38A. D.E. stands for dark exposure and L.E. stands for light exposure. (E) Immunoblots of GST-14-3-3 pulldown from HEK293T cells co-expressing HA-PDE3B with Flag-TBK1 or Flag-TBK1 K38A. Ponceau S staining shows the amount of beads used in GST-14-3-3 pulldown.

FIG. 4 shows IKKε and TBK1 phosphorylate PDE3B at serine 318, resulting in the binding of 14-3-3β. (A) Summary of sites on PDE3B phosphorylated by IKKε or TBK1 (P-sites) from mass spectrometry experiments. (B) Immunoblots of GST-14-3-3 pulldown from HEK293T cells co-expressing HA-PDE3B or HA-PDE3B S318A with Flag-TBK1. Ponceau S staining shows the amount of beads used in GST-14-3-3 pulldown. (C) GST-14-3-3 overlay on nitrocellulose membrane (top blot) and an immunoblot (IB) of whole cell lysates from HEK293T cells co-expressing HA-PDE3B or HA-PDE3B S318A with Flag-TBK1 (bottom blot). (D) cAMP levels from 3T3-L1 adipocytes expressing empty vector, HA-PDE3B, or HA-PDE3B S318A treated with or without 100 ng/ml TNFα for 16 hr followed by treatment with or without 25 μM FSK for 15 min.

FIG. 5 shows The IKKε/TBK1 inhibitor Amlexanox sensitizes β-adrenergic agonist-stimulated lipolysis in white adipose tissue in diet-induced obese mice. (A) Fold increase in serum FFA (left panel) and glycerol (right panel) levels 15 min after CL-316,243 injection in ND- or HFD-fed mice treated with amlexanox or vehicle control for 4 days. (B) Glycerol release from ex vivo epididymal (left panel) and inguinal (right panel) WATs after 1 hr pretreatment with amlexanox or vehicle. CL-316,243 treatment was started at time zero. (C) Immunoblots in inguinal WAT lysates from FIG. 5B after 60 min of CL-316,243 treatment. (D) cAMP levels in epididymal WAT 20 min after CL-316,243 (CL) or saline control injection in HFD-fed mice treated with amlexanox or vehicle control for 4 days. (E) Immunoblots in epididymal WAT 5 min after CL-316,243 or saline control injection in HFD-fed mice treated with amlexanox or vehicle control for 4 days. (F) Relative oxygen comsumption of mice in each treatment group.

### DEFINITIONS

The terms “Inhibitor of nuclear factor kappa-B kinase subunit epsilon,” “I-kappa-B kinase epsilon”, “IKKε”, and “IKKi” are used interchangeably herein to refer to the enzyme encoded by the IKBKE gene.

### DETAILED DESCRIPTION

Provided herein are methods of treating obesity and obesity-related conditions comprising the administration of combinations of IKKε/TBK1 inhibitors with beta adrenergic agonists or sympathetic nervous system activators, and pharmaceutical compositions comprising such combinations. In some embodiments, the present invention provides combinations of amlexanox or other IKKi/TBK1 inhibitors with beta-adrenergic activators (e.g., beta 2 or beta 3), or with agents that activate the sympathetic nervous system (e.g., amphetamines, phentermine).

In some embodiments, the present invention provides a method of reducing body fat or preventing increase in body fat in a subject, comprising: administering to a subject experiencing or at risk of overweight or obese body composition a therapeutically effective dose of a (i) IKKε/TBK1 inhibitor and (ii)(A) a beta adrenergic agonist or (B) sympathetic nervous system activator. In some embodiments, the administration results in reduction of or prevention of increase in body fat in the subject. In some embodiments, the subject is experiencing or is at risk of experiencing a condition such as diabetes and insulin resistance. In some embodiments, administering pharmaceutical composition results in an outcome such as: increased glucose metabolism, increased insulin receptor signaling, decreased level of insulin receptor phosphorylation, reduction in or prevention of chronic inflammation in liver, reduction in or prevention of chronic inflammation in adipose tissue, reduction in or prevention of hepatic steatosis, promotion of metabolic energy expenditure, reduction in circulating free fatty acids, and/or reduction in cholesterol.

In some embodiments, an IKKi inhibitor is a TBK1/IKKi dual inhibitor. In some embodiments, a TBK1/IKKi inhibitor is a small molecule. For example, in some embodiments, a TBK1/IKKi dual inhibitor is a 2-amino-4-(3′-cyano-4′-pyrrolidine)phenyl-pyrimidine compound or derivatives or analogues thereof (Li et al., Int J Cancer. 2013 Oct. 6. doi: 10.1002/ijc.28507; herein incorporated by reference in its entirety). In other embodiments, a TBK1/IKKi dual inhibitor is A20, TAX1BP1 (Parvatiyar et al., The Journal of Biological Chemistry, 285, 14999-15009 (2010); herein incorporated by reference in its entirety) or derivatives or analogues thereof. In some embodiments, a TBK1/IKKi inhibitor is amlexanox, a derivative or analogue thereof, or a pharmaceutically acceptable salt thereof.

Amlexanox, or 2-amino-7-isopropyl-1-azaxanthone-3-carboxylic acid; 2-amino-7-isopropyl-5-oxo-5H-chromeno[2,3-b]pyridine-3-carboxylic acid, is described in, for example, U.S. Pat. No. 4,143,042, herein incorporated by reference in its entirety. In some embodiments, the compound has the structure of Formula I:

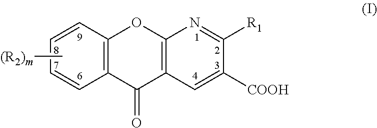

wherein R₁ is a hydrogen, alkyl, phenyl, carboxyl, hydroxyl, alkoxy, or amino group, which may be unsubstituted or substituted by one alkyl; m is 0, 1 or 2; and R₂ is alkyl, alkoxy, halogen, nitro, hydroxy, carboxyl, butadienylene (—CH═CH—CH═CH—), which forms a benzene ring with any adjacent carbon atoms or amino group, which may be unsubstituted or substituted by at least one alkyl, and their physiologically acceptable salts. The substituents designated in each of the above-mentioned formulae may be substituted at optional position or positions of the 6-, 7-, 8-, or 9-positions of the azaxanthone ring.

In Formula (I), the alkyl group represented by R₁ and R₂ may be any of straight-chain, branched, or cyclic alkyl group having 1 to 6 carbon atoms. Typical examples of the alkyl group may be methyl, ethyl, propyl, isopropyl, butyl, isobutyl, sec-butyl, tert-butyl, pentyl, cyclopentyl, hexyl, cyclohexyl, etc.

The alkoxy group represented by R₁ and R₂ may, for example, be that having 1 to 4 carbon atoms in the alkyl moieties, such as methoxy, ethoxy, propoxy, isopropoxy, butoxy, etc.

The mono-alkyl substituted amino group represented by R₁ may be that having 1 to 3 carbon atoms in the alkyl moieties, such as methylamino, ethylamino, propylamino, or isopropylamino. The halogen represented by R₂ may be chlorine, bromine, iodine, or fluorine.

The alkyl substituted amino group represented by R₂ includes mono- or di-alkyl substituted ones whose alkyl moiety is that having 1 to 3 carbon atoms, e.g., methylamino, ethylamino, propylamino, isopropylamino, dimethylamino, diethylamino, or dipropylamino

The compound of general Formula (I) can be converted to the corresponding organic amine salts, alkali metal salts, or ammonium salts by reacting (I) in the per se conventional manner with an organic amine (e.g., ethanolamine, diethanolamine, dl-methylephedrin, 1-(3,5-dihydroxyphenyl)-L-isopropylaminoethanol, isoproterenol, dextromethorphan, hetrazan (diethylcarbamazine), diethylamine, triethylamine, etc.), an alkali metal hydroxide (e.g., sodium hydroxide, potassium hydroxide, etc.) or ammonia, for example by mixing them together and heating in a suitable solvent.

In some embodiments, a sympathomimetic agent (e.g., small molecule, peptide, RNA, etc.) is provided that acts upon a beta adrenoceptor, having the opposite effect of a beta blocker. In some embodiments, a beta adrenergic receptor agonist is provided. In some embodiments, beta adrenergic receptor agonists mimic the action of epinephrine and norepinephrine signaling. In some embodiments, a beta adrenergic receptor agonist activates one or more of β1, β2, and β receptors.

In some embodiments, a β1 agonist selected from Dobutamine, Isoproterenol (β1 and β2), Xamoterol, epinephrine, etc. is provided. Other suitable β1 agonists are within the scope of the invention.

In some embodiments, a β2 agonist selected from salbutamol (albuterol in USA) levosalbutamol (Levalbuterol in USA), fenoterol, formoterol, isoproterenol (β1 and β2), metaproterenol, salmeterol, terbutaline, clenbuterol, isoetarine, pirbuterol, procaterol, ritodrine, epinephrine, etc. is provided. Other suitable β2 agonists are within the scope of the invention.

In some embodiments, any suitable β agonists (e.g., Mirabegron) are within the scope of the invention.

In some embodiments, a beta adrenergic receptor agonist is select from the list including, but not limited to salbutamol (albuterol, Ventolin), levosalbutamol (levalbuterol, Xopenex), terbutaline (Bricanyl), pirbuterol (Maxair), procaterol, clenbuterol, metaproterenol (Alupent), fenoterol, bitolterol mesylate, ritodrine, isoprenaline, salmeterol (Serevent Diskus), formoterol (Foradil, Symbicort), bambuterol, clenbuterol, indacaterol, arbutamine, befunolol bromoacetylalprenololmenthane, broxaterol, cimaterol, cirazoline, denopamine, dopexamine, etilefrine, hexoprenaline, higenamine, isoxsuprine, mabuterol, methoxyphenamine, nylidrin, oxyfedrine, prenalterol, ractopamine, reproterol, rimiterol, tretoquinol, tulobuterol, zilpaterol, zinterol, etc.

In some embodiments, a sympathetic nervous system activator is provided. Such agents may activate the sympathetic nervous system by any suitable mechanism (e.g., acting on, increasing the release of, or inhibiting reuptake of one or more neurotransmitters (e.g., norepinephrine, serotonin and dopamine epinephrine and/or adrenaline), acting as an adrenergic receptor agonist, etc.). Suitable sympathetic nervous system activators may be selected from Benzodiazepines (e.g., Diazepam (Valium), clonazepam (Klonopin), lorazepam (Ativan), temazepam (Restoril), flunitrazepam (Rohypnol), triazolam (Halcion), alprazolam (Xanax), etc.), Amphetamines (e.g., Amphetamine (Adderall), methamphetamine (Desoxyn), methylphenidate (Ritalin), phentermine, 4-methylaminorex, phenmetrazine (Preludin), methcathinone, fenfluramine (Pondimin, Fen-Phen), dexfenfluramine (Redux), pseudoephedrine (Sudafed), ephedrine, phenylpropanolamine (old Triaminic), phenylephrine (Sudafed PE), etc.), phentermine, topiramate, etc. Other suitable sympathetic nervous system activators are within the scope of the invention.

In some embodiments, the present invention finds use in the treatment or prevention of overweight and obesity. The most widely accepted clinical definition of obesity is the World Health Organization (WHO) criteria based on BMI. Under this convention for adults, grade 1 overweight (commonly and simply called overweight) is a BMI of 25-29.9 kg/m². Grade 2 overweight (commonly called obesity) is a BMI of 30-39.9 kg/m². Grade 3 overweight (commonly called severe or morbid obesity) is a BMI greater than or equal to 40 kg/m². The surgical literature often uses a different classification to recognize particularly severe obesity. In this setting, a BMI greater than 40 kg/m² is described as severe obesity, a BMI of 40-50 kg/m² is termed morbid obesity, and a BMI greater than 50 kg/m² is termed super obese. The definition of obesity in children involves BMIs greater than the 85th (commonly used to define overweight) or the 95th (commonly used to define obesity) percentile, respectively, for age-matched and sex-matched control subjects. Secondary causes of obesity include but are not limited to hypothyroidism, Cushing syndrome, insulinoma, hypothalamic obesity, polycystic ovarian syndrome, genetic syndromes (eg, Prader-Willi syndrome, Alstrom syndrome, Bardet-Biedl syndrome, Cohen syndrome, Borjeson-Forssman-Lehmann syndrome, Frohlich syndrome), growth hormone deficiency, oral contraceptive use, medication-induced obesity (e.g., phenothiazines, sodium valproate, carbamazepine, tricyclic antidepressants, lithium, glucocorticoids, megestrol acetate, thiazolidine diones, sulphonylureas, insulin, adrenergic antagonists, serotonin antagonists [especially cyproheptadine]), eating disorders (especially binge-eating disorder, bulimia nervosa, night-eating disorder), hypogonadism, pseudohypoparathyroidism, and obesity related to tube feeding. In some embodiments, pharmaceutical combinations and treatments described herein find use in the treatment of one or more of the aforementioned secondary causes of obesity.

In some embodiments, a subject is tested to assess the presence, the absence, or the level of a disease or condition (e.g., obesity and/or a related disorder, including, but not limited to insulin resistance, diabetes, steatosis, nonalcoholic steatotic hepatitis, and atherosclerosis), e.g., by assaying or measuring a biomarker, a metabolite, a physical symptom, an indication, etc., to determine the risk of or the presence of obesity and/or a related disorder, including, but not limited to insulin resistance, diabetes, steatosis, nonalcoholic steatotic hepatitis, and atherosclerosis, and thereafter the subject is treated with a pharmaceutical combination described herein based on the outcome of the test. In some embodiments, a patient is tested, treated, and then tested again to monitor the response to therapy. In some embodiments, cycles of testing and treatment may occur without limitation to the pattern of testing and treating (e.g., test/treat, test/treat/test, test/treat/test/treat, test/treat/test/treat/test, test/treat/treat/test/treat/treat, etc.), the periodicity, or the duration of the interval between each testing and treatment phase.

It is generally contemplated that the compositions and/or pharmaceutical combinations according to the technology provided are formulated for administration to a mammal, and especially to a human with a condition that is responsive to the administration of such compounds. Therefore, where contemplated compounds are administered in a pharmacological composition or combination, it is contemplated that a formulation may be in the form of an admixture with a pharmaceutically acceptable carrier. For example, contemplated compounds and combinations can be administered orally as pharmacologically acceptable salts, or intravenously in a physiological saline solution (e.g., buffered to a pH of about 7.2 to 7.5). Conventional buffers such as phosphates, bicarbonates, or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

In certain pharmaceutical dosage forms, prodrug forms of contemplated compounds may be formed for various purposes, including reduction of toxicity, increasing the organ or target cell specificity, etc. Among various prodrug forms, acylated (acetylated or other) derivatives, pyridine esters, and various salt forms of the present compounds are preferred. One of ordinary skill in the art will recognize how to readily modify the present compounds to prodrug forms to facilitate delivery of active compounds to a target site within the host organism or patient. One of ordinary skill in the art will also take advantage of favorable pharmacokinetic parameters of the prodrug forms, where applicable, in delivering the present compounds to a targeted site within the host organism or patient to maximize the intended effect of the compound. Similarly, it should be appreciated that contemplated compounds may also be metabolized to their biologically active form, and all metabolites of the compounds herein are therefore specifically contemplated. In addition, contemplated compounds (and combinations thereof) may be administered in combination with yet further agents for treating obesity and related disorders, including, but not limited to insulin resistance, diabetes, steatosis, nonalcoholic steatotic hepatitis, and atherosclerosis.

With respect to administration to a subject, it is contemplated that the compounds and/or combinations be administered in a pharmaceutically effective amount. One of ordinary skill recognizes that a pharmaceutically effective amount varies depending on the therapeutic agent used, the subject's age, condition, and sex, and on the extent of the disease in the subject. Generally, the dosage should not be so large as to cause adverse side effects, such as hyperviscosity syndromes, pulmonary edema, congestive heart failure, and the like. The dosage can also be adjusted by the individual clinician to achieve the desired therapeutic goal.

As used herein, the actual amount encompassed by the term “pharmaceutically effective amount” will depend on the route of administration, the type of subject being treated, and the physical characteristics of the specific subject under consideration. These factors and their relationship to determining this amount are well known to skilled practitioners in the medical, veterinary, and other related arts. This amount and the method of administration can be tailored to achieve optimal efficacy but will depend on such factors as weight, diet, concurrent medication, and other factors that those skilled in the art will recognize.

In some embodiments, a single pharmaceutical composition comprising both a (i) IKKε/TBK1 inhibitor and (ii) (A) a beta adrenergic agonist or (B) sympathetic nervous system activator is provided. In other embodiments, separate pharmaceutical compositions are administered, one comprising an IKKε/TBK1 inhibitor and another comprising a beta adrenergic agonist and/or sympathetic nervous system activator. Dosing and scheduling of administration of separate pharmaceutical compositions may be determined and/or adjusted jointly or separately.

The dosage amount and frequency are selected to create an effective level of the compound without substantially harmful effects. When administered orally or intravenously, the dosage will generally range from 0.001 to 10,000 mg/kg/day or dose (e.g., 0.01 to 1000 mg/kg/day or dose; 0.1 to 100 mg/kg/day or dose, 1 to 100 mg/kg/day or dose, or amounts therein).

In some embodiments, a single dose is administered to a subject. In other embodiments, multiple doses are administered over two or more time points, separated by hours, days, weeks, etc. In some embodiments, pharmaceutical compositions are administered over a long period of time (e.g., chronically), for example, for a period of months or years (e.g., 1, 2, 3, 4, 5, 6, 7, 8, 9, 10, 11, 12, or more months or years). In such embodiments, pharmaceutical compositions may be taken on a regular scheduled basis (e.g., daily, weekly, etc.) for the duration of the extended period.

Methods of administering a pharmaceutically effective amount include, without limitation, administration in parenteral, oral, intraperitoneal, intranasal, topical, sublingual, rectal, and vaginal forms. Parenteral routes of administration include, for example, subcutaneous, intravenous, intramuscular, intrastemal injection, and infusion routes. In some embodiments, amlexanox, a derivative thereof, or a pharmaceutically acceptable salt thereof, is administered orally.

Pharmaceutical compositions preferably comprise one or more compounds of the present technology associated with one or more pharmaceutically acceptable carriers, diluents, or excipients. Pharmaceutically acceptable carriers are known in the art such as those described in, for example, Remingtons Pharmaceutical Sciences, Mack Publishing Co. (A. R. Gennaro edit. 1985), explicitly incorporated herein by reference for all purposes.

Accordingly, in some embodiments, the composition (comprising one agent or a pharmaceutical composition) is formulated as a tablet, a capsule, a time release tablet, a time release capsule; a time release pellet; a slow release tablet, a slow release capsule; a slow release pellet; a fast release tablet, a fast release capsule; a fast release pellet; a sublingual tablet; a gel capsule; a microencapsulation; a transdermal delivery formulation; a transdermal gel; a transdermal patch; a sterile solution; a sterile solution prepared for use as an intramuscular or subcutaneous injection, for use as a direct injection into a targeted site, or for intravenous administration; a solution prepared for rectal administration; a solution prepared for administration through a gastric feeding tube or duodenal feeding tube; a suppository for rectal administration; a liquid for oral consumption prepared as a solution or an elixir; a topical cream; a gel; a lotion; a tincture; a syrup; an emulsion; or a suspension.

In some embodiments, the time release formulation is a sustained-release, sustained-action, extended-release, controlled-release, modified release, or continuous-release mechanism, e.g., the composition is formulated to dissolve quickly, slowly, or at any appropriate rate of release of therapeutic agents over time.

In some embodiments, the pharmaceutical preparations and/or formulations of the technology are provided in particles. Particles as used herein means nano or microparticles (or in some instances larger) that can consist in whole or in part of the therapeutic agents as described herein. The particles may contain the preparations and/or formulations in a core surrounded by a coating, including, but not limited to, an enteric coating. The preparations and/or formulations also may be dispersed throughout the particles. The preparations and/or formulations also may be adsorbed into the particles. The particles may be of any order release kinetics, including zero order release, first order release, second order release, delayed release, sustained release, immediate release, and any combination thereof, etc. The particle may include, in addition to the preparations and/or formulations, any of those materials routinely used in the art of pharmacy and medicine, including, but not limited to, erodible, nonerodible, biodegradable, or nonbiodegradable material or combinations thereof. The particles may be microcapsules which contain the formulation in a solution or in a semi-solid state. The particles may be of virtually any shape.

Both non-biodegradable and biodegradable polymeric materials can be used in the manufacture of particles for delivering the preparations and/or formulations. Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26: 581-587, the teachings of which are incorporated herein by reference. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly (isobutyl methacrylate), poly(hexylmethacrylate), poly(isodecyl methacrylate), poly(lauryl methacrylate), poly(phenylmethacrylate), poly(methyl acrylate), poly(isopropyl acrylate), poly(isobutyl acrylate), and poly(octadecyl acrylate).

In some embodiments, the pharmaceutical compositions are formulated with a buffering agent. The buffering agent may be any pharmaceutically acceptable buffering agent. Buffer systems include citrate buffers, acetate buffers, borate buffers, and phosphate buffers. Examples of buffers include citric acid, sodium citrate, sodium acetate, acetic acid, sodium phosphate and phosphoric acid, sodium ascorbate, tartartic acid, maleic acid, glycine, sodium lactate, lactic acid, ascorbic acid, imidazole, sodium bicarbonate and carbonic acid, sodium succinate and succinic acid, histidine, and sodium benzoate and benzoic acid.

### EXPERIMENTAL

Obesity produces a chronic inflammatory state involving the NFκB pathway, resulting in persistent elevation of the noncanonical IκB kinases IKKi and TBK1. Experiments conducted during development of embodiments of the present invention demonstrate that these kinases attenuate β-adrenergic signaling in white adipose tissue. Treatment of 3T3-L1 adipocytes with specific inhibitors of these kinases restored β-adrenergic signaling and lipolysis attenuated by TNFα and Poly (I:C). Conversely, overexpression of the kinases reduced induction of Ucp1, lipolysis, cAMP levels, and phosphorylation of hormone sensitive lipase in response to isoproterenol or forskolin. Noncanonical IKKs reduce catecholamine sensitivity by phosphorylating and activating the major adipocyte phosphodiesterase PDE3B. In vivo inhibition of these kinases by treatment of obese mice with the drug amlexanox reversed obesity-induced catecholamine resistance, and restored PKA signaling in response to injection of a β-3 adrenergic agonist. These studies indicate that by reducing production of cAMP in adipocytes, IKKε and TBK1 contribute to the repression of energy expenditure during obesity.

### Example 1

### IKKε and TBK1 Overexpression Decrease Sensitivity to the β-Adrenergic/cAMP Pathway in 3T3-L1 Adipocytes

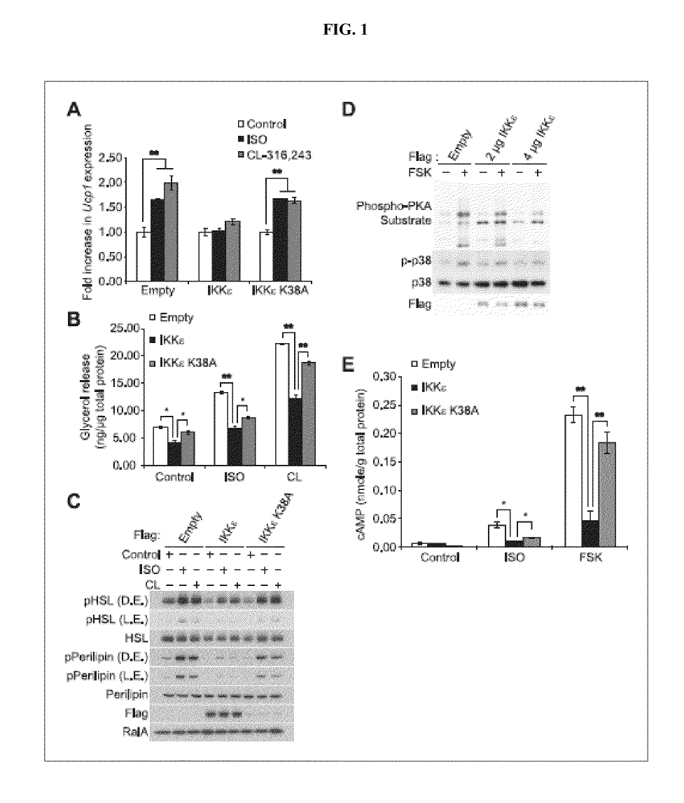

Sympathetic activation of adipose tissue is involved in maintaining energy balance by stimulating lipolysis and fat oxidation (Coppack et al., 1994; Langin, 2006; Festuccia et al., 2011; herein incorporated by reference in their entireties). Activation of β-adrenergic signaling by either β-adrenergic agonists or cold exposure in white and brown adipose tissue initiates a cascade of events through cyclic AMP (cAMP), culminating in the transcriptional upregulation of Ucp1, which results in increased proton leak and energy expenditure (Himms-Hagen et al., 2000; Cao et al., 2004; Yehuda-Shnaidman et al., 2010; herein incorporated by reference in their entireties). Compared to wild-type (WT) controls, IKKε-deficient mice exhibit increased energy expenditure while on a high fat diet (HFD), accompanied by increased expression of Ucp1 in white adipose depots (Chiang et al., 2009; herein incorporated by reference in its entirety). Increased energy expenditure in IKKε-deficient mice was only seen in HFD-fed mice (Chiang et al., 2009; herein incorporated by reference in its entirety), indicating that upon induction of IKKε during obesity, the kinase represses an increased adaptive thermogenic response to overnutrition. This effect was further analyzed by overexpressing IKKε in 3T3-L1 adipocytes and examining Ucp1 gene expression after treatment with the non-selective β-adrenergic agonist, isoproterenol (ISO), or the β3-adrenergic agonist, CL-316,243. Fold difference in Ucp1 gene expression was calculated by normalization of relative Ucp1 mRNA levels in treated relative to control samples. Treatment of empty vector-expressing cells with ISO or CL-316,243 resulted in a 1.6-fold or twofold increase in Ucp1 mRNA levels, respectively (FIG. 1A). The induction of Ucp1 gene expression in response to ISO or CL-316,243 was blunted when WT IKKε was overexpressed in these cells. However, expression of the kinase-inactive mutant of IKKεK38A (Fitzgerald et al., 2003; herein incorporated by reference in its entirety) was less effective, but still modestly repressed Ucp1 expression.

In addition to increased Ucp1 expression, IKKε knockout mice also exhibited increased lipolysis and fat oxidation (Chiang et al., 2009; herein incorporated by reference in its entirety), suggesting that decreased lipolysis in adipose tissue from obese mice might result in part from increased expression of IKKε and TBK1 (Chiang et al., 2009; herein incorporated by reference in its entirety). The obesity-dependent increase was modeled in the noncanonical IKKs by overexpressing IKKε in 3T3-L1 adipocytes, followed by assay of glycerol release in response to ISO or CL-316,243. Although both isoproterenol and CL-316,243 increased lipolysis in empty vector-expressing cells, overexpression of WT IKKε reduced the lipolytic effects of isoproterenol and CL-316,243 by greater than 40%, and also reduced basal glycerol release (FIG. 1B). The reduction in lipolysis by IKKε overexpression was accompanied by dramatically reduced phosphorylation of HSL and perilipin in response to ISO or CL-316,243 (FIG. 1C). Expression of the catalytically inactive kinase was less effective in blocking lipolytic signaling, although the levels of protein achieved by overexpression were lower compared to the WT kinase (FIG. 1B,C, FIG. 1). Overexpression of TBK1 reduced phosphorylation of HSL in response to isoproterenol or the adenylyl cyclase activator, forskolin (FIG. 1). Identical results were obtained when IKKε was overexpressed in 3T3-L1 adipocytes stimulated with forskolin (FIG. 1D), as detected by western blotting with an anti-phospho-PKA substrate motif antibody.

Overexpression of IKKε also repressed the phosphorylation of p38 (p-p38) in response to forskolin (FIG. 1D) or isoproterenol (FIG. 1), whereas overexpression of IKKε K38A was without effect (FIG. 1). While glycerol release is likely the result of changes in HSL and perilipin phosphorylation, it is important to note that we have not directly assayed whether re-esterification of glycerol intermediates are also affected. Taken together, these data indicate that similar to what is observed in obesity, overexpression of IKKε or TBK1 can repress lipolytic signaling. It is contemplated that the partial effectiveness of the kinase-inactive mutants reflects their activation of endogenous IKKε or TBK1 kinases due to dimerization (Larabi et al., 2013; Tu et al., 2013; herein incorporated by reference in their entireties).

Since PKA signaling is responsible for Ucp1 induction in response to catecholamines (Klein et al., 2000; Cao et al., 2001; herein incorporated by reference in their entireties); experiments were conducted during development of embodiments of the present invention to investigate IKKε and TBK1 induced reduction of β-adrenergic sensitivity of adipocytes by decreasing cAMP levels. IKKε overexpression in 3T3-L1 adipocytes reduced by greater than 80% the increase in cAMP levels produced by both isoproterenol and forskolin, whereas overexpression of IKKε K38A did not (FIG. 1E). Previous studies have shown that decreased sensitivity to adrenergic stimuli in adipose tissue can result from reduced (3-adrenergic receptors (Reynisdottir et al., 1994; herein incorporated by reference in its entirety) or increased expression of α2-adrenergic receptors (Stich et al., 2002; herein incorporated by reference in its entirety).

### Example 2

### Prolonged Treatment with TNFα Decreases the Sensitivity of Adipocytes to β-Adrenergic Stimulation in a Manner Dependent on the Activity of IKKε and TBK1

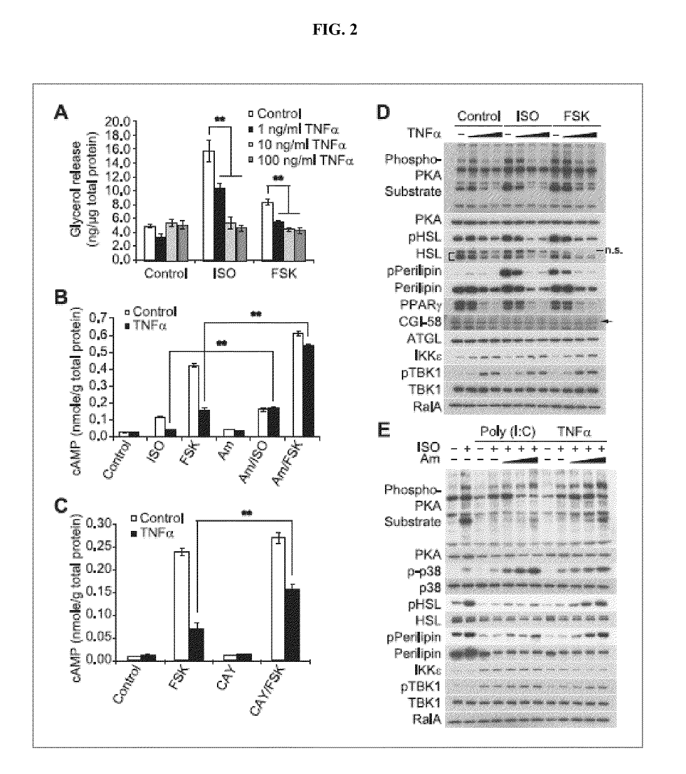

Obesity is accompanied by infiltration of proinflammatory macrophages into adipose tissue; these cells secrete inflammatory cytokines, such as TNFα, which generate insulin resistance by stimulating catabolic pathways (Hotamisligil, 2006; Lumeng et al., 2007; Ye and Keller, 2010; Ouchi et al., 2011; herein incorporated by reference in their entireties). Although TNFα is known to increase lipolysis in adipocytes (Zhang et al., 2002; Souza et al., 2003; Green et al., 2004; Plomgaard et al., 2008; herein incorporated by reference in their entireties), there is also evidence of a counterinflammatory response in obesity that may serve to repress energy expenditure (Gregor and Hotamisligil, 2011; Saltiel, 2012; Calay and Hotamisligil, 2013; Reilly et al., 2013; herein incorporated by reference in their entireties). Experiments were conducted during development of embodiments of the present invention using TNFα to model the inflammatory milieu of obese adipose tissue in cell culture to determine whether the cytokine might also regulate β-adrenergic signaling in this context. While short-term treatment with TNFα augmented the increase in cAMP produced by forskolin treatment, this effect declined after 12 hr. After 24 hr of exposure, TNFα inhibited the production of the second messenger produced by forskolin (FIG. 2). Thus, the catabolic effects of the proinflammatory cytokine TNFα in adipocytes are transient and followed by an inhibitory phase.

Treatment of 3T3-L1 adipocytes with TNFα for 24 hr induced the expression of IKKε and increased TBK1 phosphorylation at the active site in a manner that was dependent on the activity of IKKβ and the NFκB pathway (Reilly et al., 2013; herein incorporated by reference in its entirety). Experiments were conducted during development of embodiments of the present invention to determine whether the repression of β-adrenergic sensitivity produced by longer-term treatment with TNFα is due to increased activity of the noncanonical IKKs. Long-term treatment with TNFα repressed the induction of Ucp1 gene expression in response to β-adrenergic stimuli (FIG. 2), whereas the expression of IKKε mRNA (Ikbke) was upregulated. Treatment of 3T3-L1 adipocytes with TNFα for 24 hr decreased glycerol release in response to both isoproterenol and forskolin in a dose-dependent manner (FIG. 2A). TNFα treatment also decreased isoproterenol- and forskolin-stimulated cAMP production; an effect that was largely rescued by preincubation of cells with the selective, but structurally unrelated inhibitors of IKKε and TBK1, amlexanox (FIG. 2B) (Reilly et al., 2013; herein incorporated by reference in its entirety) or CAY10576 (FIG. 2C) (Bamborough et al., 2006; herein incorporated by reference in its entirety). Isoproterenol-stimulated β-adrenergic signaling was also decreased by treatment of cells with TNFα (FIG. 2D), as manifested by decreased phosphorylation of HSL, perilipin, and other proteins recognized by the PKA substrate motif antibody, whereas IKKε expression was concurrently upregulated and TBK1 phosphorylation was increased by the treatment with TNFα. Pretreatment of 3T3-L1 adipocytes with amlexanox also blocked the inhibitory effect of TNFα on isoproterenol-stimulated β-adrenergic signaling, as determined by western blotting with an anti-phospho-PKA substrate motif antibody, antiphospho-HSL, and anti-phospho-perilipin antibodies (FIG. 2E). Phosphorylation of p38 in response to isoproterenol was also dramatically augmented by amlexanox in a dose-dependent manner. Previous studies showed that the toll-like receptor 3 (TLR3) agonist, Poly (I:C), results in the direct activation of IKKε and TBK1 (Hemmi et al., 2004; Clark et al., 2009; Clark et al., 2011; herein incorporated by reference in their entireties). Similar to TNFα, treatment of 3T3-L1 adipocytes with Poly (I:C) simultaneously reduced stimulation of cAMP production, lipolysis and phosphorylation in response to β-adrenergic stimulation (FIG. 2), and the inhibitory effects of Poly (I:C) on the sensitivity to isoproterenol stimulation were partially restored by amlexanox pretreatment, but not to the extent that was observed with TNFα treatment (FIG. 2E). These results indicate that obesity-associated inflammation leads to the activation of IKKε and TBK1, which produces reduced sensitivity of adipocytes to β-adrenergic stimulation.

### Example 3

### IKKε and TBK1 Reduce cAMP Levels Through Activation of PDE3B

cAMP levels can also be regulated by phosphodiesterases, which cleave the second messenger and in the process dampen cAMP-dependent signals. Phosphodiesterase 3B (PDE3B) is the major PDE isoform expressed in adipocytes (Zmuda-Trzebiatowska et al., 2006; herein incorporated by reference in its entirety). Genetic ablation or pharmacological inhibition of PDE3B in cells and in vivo revealed an important role for the enzyme in lipid and glucose metabolism (Choi et al., 2006; Berger et al., 2009; Degerman et al., 2011; herein incorporated by reference in their entireties). Phosphorylation and activation of PDE3B by insulin in adipocytes is thought to be mediated by Akt, and cAMP itself acts as a negative feedback regulator of its own levels by promoting PKA-dependent phosphorylation and activation of PDE3B (Degerman et al., 2011; herein incorporated by reference in its entirety).

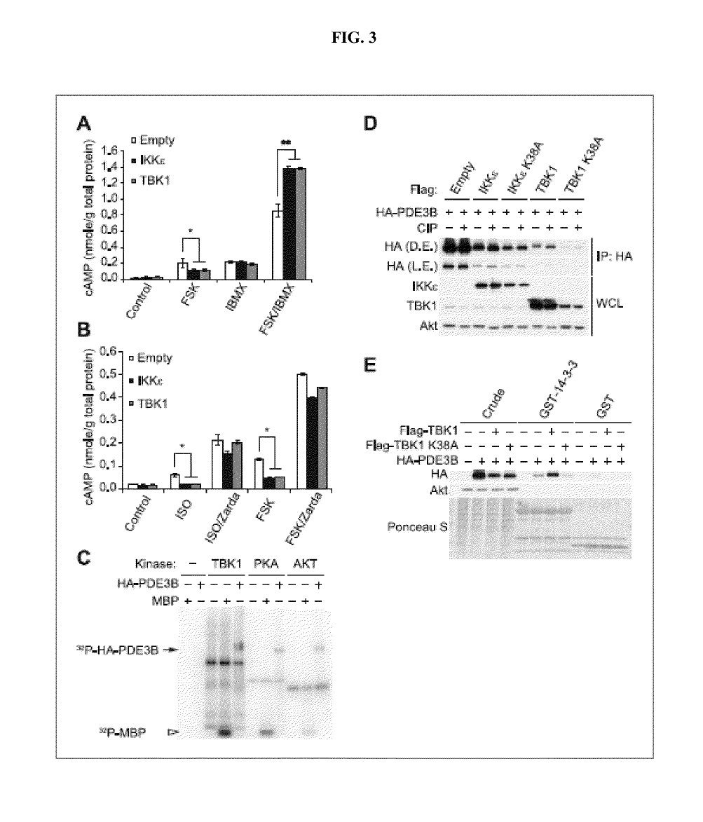

Experiments conducted during development of embodiments of the present invention demonstrated that cAMP production was impaired in forskolin or isoproterenol-stimulated 3T3-L1 adipocytes overexpressing IKKε (FIG. 1E), therefore additional experiments were conducted to determine whether noncanonical IKKs might desensitize adrenergic stimulation through increased activity of PDE3B in adipocytes. Pretreatment with a nonspecific phosphodiesterase inhibitor, IBMX, in 3T3-L1 adipocytes expressing IKKε or TBK1 rescued the full stimulation of cAMP production in response to forskolin (FIG. 3A). Interestingly, the selective PDE3B and PDE4 inhibitor, Zardaverine (Schudt et al., 1991; herein incorporated by reference in its entirety), also blocked the inhibitory effects of IKKε and TBK1 overexpression on cAMP levels in response to isoproterenol and forskolin in 3T3-L1 adipocytes (FIG. 3B), suggesting an important role for PDE3B as a target of the noncanonical IKKs.

It was next examined whether IKKε and TBK1 directly phosphorylate PDE3B to regulate cAMP levels. Recombinant TBK1, Akt and PKA were incubated in vitro with [γ-32P]ATP and purified PDE3B as a substrate. Phosphorylation was assessed by SDS-PAGE followed by autoradiography. TBK1 directly catalyzed the phosphorylation of PDE3B; phosphorylation was also produced by incubation with Akt and PKA, as previously reported (Kitamura et al., 1999; Palmer et al., 2007; herein incorporated by reference in their entireties) (FIG. 3C). IKKε also catalyzed this phosphorylation in vitro. This increase in phosphorylation produced by in vitro incubation with TBK1, IKKε and PKA was also detected when PDE3B was blotted with antibodies that recognize the 14-3-3 binding motif (FIG. 3). When purified PDE3B was incubated with the same amount of recombinant TBK1 and canonical IKKβ kinases in vitro, phosphorylation of PDE3B by IKKβ was barely detectable, indicating a level of specificity in which PDE3B is a better target of the noncanonical IKKs (FIG. 3). This phosphorylation was dose-dependent with respect to ATP (FIG. 3).

To determine whether IKKε can phosphorylate PDE3B in cells, IKKε and its inactive mutant K38A were co-expressed with HA-tagged PDE3B in HEK293T cells, followed by immunoprecipitation (IP) with anti-HA antibodies. Expression of IKKε in cells caused a shift in electrophoretic mobility of PDE3B, and this shift was not detected when IKKε K38A was expressed (FIG. 3). Phosphorylation of PDE3B was also detected after expression of IKKε but not its kinase-inactive mutant K38A in cells, as detected by blotting with antibodies that recognize the 14-3-3 binding motif. To determine whether this molecular shift was dependent on phosphorylation of PDE3B, HA-PDE3B was co-expressed in Cos-1 cells along with IKKε, TBK1 or their kinase inactive mutants, and HA immunoprecipitates were treated with or without calf intestinal phosphatase (CIP). Expression of both of the wild-type kinases reduced the electrophoretic mobility of PDE3B, which could be reversed by treatment with the phosphatase (FIG. 3D, compare lane 3, 7 to lane 4, 8). Neither of the kinase-inactive mutants had an effect (FIG. 3D, compare lane 5, 9 to lane 6, 10).

Previous studies suggested that IKKε and TBK1 bind to their respective substrates through a sequence that includes a ubiquitin-like domain (ULD) proximal to their kinase domain. This domain is highly conserved among the IKK family members, and is 49% identical between IKKε and TBK1 (Ikeda et al., 2007; May et al., 2004; herein incorporated by reference in their entireties). To confirm that PDE3B is a bona fide substrate of IKKε and TBK1, a GST-ULD domain fusion protein was prepared from TBK1 and incubated this fusion protein with 3T3-L1 adipocyte lysates. The fusion protein specifically precipitated endogenous PDE3B from these lysates (FIG. 3). To explore further the interaction of these two proteins, WT TBK1 and its K38A mutant were co-expressed with HA-tagged PDE3B in HEK293T cells, and immunoprecipitated the protein with anti-HA antibodies. Kinase-inactive TBK1 was preferentially co-immunoprecipitated with PDE3B, whereas the interaction of PDE3B with WT TBK1 was barely detectable (FIG. 3). These data indicate that TBK1 and IKKε associate with substrates such as PDE3B, and subsequently dissociate upon phosphorylation.

Too test further the role of PDE3B phosphorylation by IKKε and TBK1 in initiating its interaction with 14-3-3β, a GST-14-3-3β fusion protein was prepared which was incubated with lysates from HEK293T cells co-expressing TBK1 with PDE3B. PDE3B was preferentially pulled down by GST-14-3-3β after phosphorylation by TBK1 but not by its inactive K38A mutant, whereas GST beads alone enriched neither PDE3B nor its phosphorylated form (FIG. 3E).

### Example 4

### IKKε and TBK1 Phosphorylate PDE3B at Serine 318, Resulting in the Binding of 14-3-3β

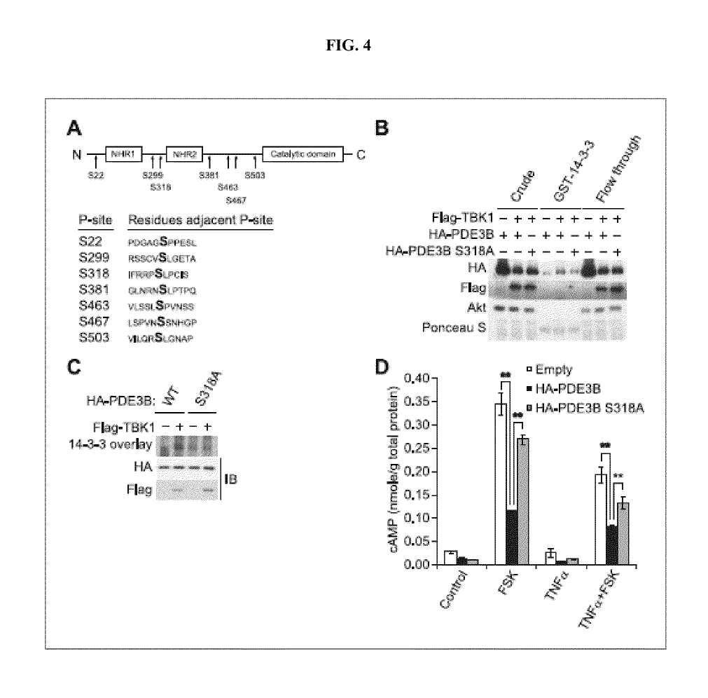

To evaluate the regulatory role of PDE3B phosphorylation by IKKε and TBK1, we determined which sites are phosphorylated. HA-PDE3B was co-expressed in Cos-1 cells with IKKε and TBK1, and phosphorylated PDE3B was enriched by IP with anti-HA antibodies. Phosphorylation sites on human PDE3B were then determined by LC-MS/MS mass spectrometry. This analysis revealed that serines 22, 299, 318, 381, 463, 467, and 503 were phosphorylated by both kinases; there were no differences between the kinases (FIG. 4A). Interestingly, the phosphorylation profile of PDE3B matched neither known Akt or PKA profiles (Lindh et al., 2007). However, phosphorylation on serine 299 and serine 318 had previously been identified on mouse PDE3B (residues equivalent to Serine 277 and 296 in mouse PDE3B) in adipocytes and hepatocytes in response to both insulin and forskolin (Lindh et al., 2007). While several serine residues are known to be phosphorylated on PDE3B in response to stimuli, serine 318 (human) is the best characterized. This residue resides in a consensus phosphorylation sequence for both Akt and PKA, and also serves as a consensus 14-3-3 binding motif once phosphorylated (Lindh et al., 2007; Palmer et al., 2007). We thus created a Ser318Ala (S318A) mutant of PDE3B, and examined its interaction with a GST-14-3-3β fusion protein or by GST-14-3-3 overlay assay. Interestingly, despite incubation with TBK1, the phospho-defective, S318A mutant of PDE3B, did not specifically interact with GST-14-3-3β, whereas the wild-type protein did (FIG. 4B,C). In a GST pull-down assay, the molecular shift of PDE3B S318A was still detected by western blot (FIG. 4B), indicating that phosphorylation of PDE3B by TBK1 on other sites still occurred, but were not crucial for 14-3-3β binding.

To examine the functional importance of the phosphorylation of PDE3B at Serine 318, we overexpressed WT PDE3B and its S318A mutant in 3T3-L1 adipocytes, and tested the response of the cells to TNFα. Overexpression of WT PDE3B in cells reduced the attenuation of forskolin-stimulated cAMP production and phosphorylation of HSL produced by TNFα, whereas PDE3B S318A was ineffective (FIG. 4D, FIG. 4—figure supplement 1A,B). These data suggest that although IKKε and TBK1 can phosphorylate PDE3B on several sites, serine 318 may be particularly important in the regulation of phosphodiesterase function by promoting the interaction between PDE3B and 14-3-3p. More importantly, this residue is the major site mediating the negative effects of IKKε and TBK1 on sensitivity of adipocytes to β-adrenergic stimulation.

### Example 5

### The IKKc/TBK1 Inhibitor Amlexanox Sensitizes β-Adrenergic Agonist Stimulated Lipolysis in White Adipose Tissue in Diet-Induced Obese Mice

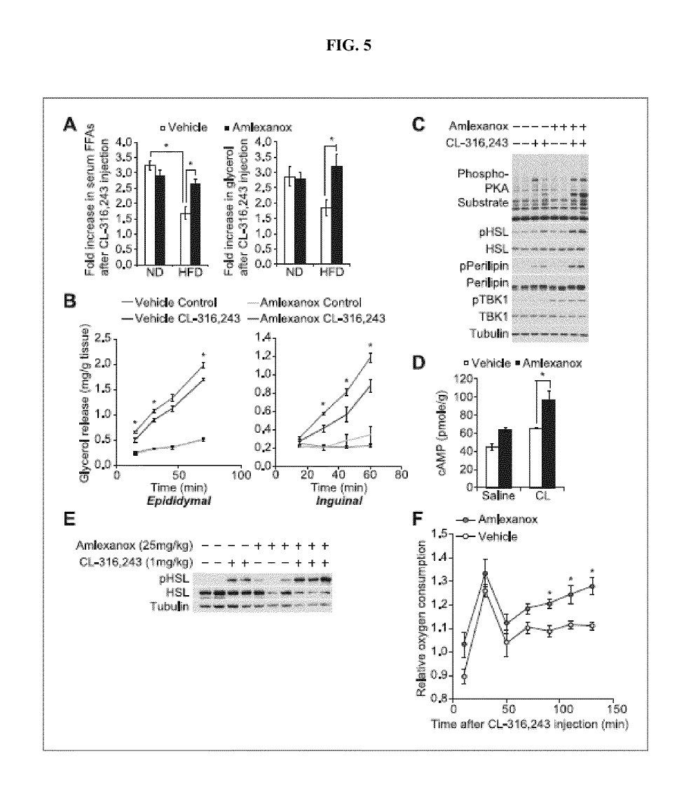

To test the functional importance of the noncanonical IKKs in maintaining energy balance in vivo, experiments were conducted during development of embodiments of the present invention to investigate whether the administration of a selective inhibitor of IKKε and TBK1, amlexanox, can reverse diet-induced catecholamine resistance in rodentsMice were fed a high fat or normal diet, treated them with amlexanox by oral gavage for 4 days (prior to the point when weight loss is seen), and then gave a single intraperitoneal (IP) injection of the β3-adrenergic agonist CL-316,243. Injection of CL-316,243 stimulated a threefold increase in serum FFA and glycerol levels in both vehicle and amlexanox-treated mice on normal diet (ND). The effect of CL-316,243 to increase serum FFAs was significantly attenuated in HFD-fed, vehicle-treated mice. However, HFD-fed mice treated with amlexanox responded like normal diet mice, despite the fact that they were weight matched with control HFD-fed mice (FIG. 5A). The fold increase in serum glycerol levels was also significantly higher in amlexanox-treated HFD mice, as compared to vehicle-treated HFD-fed mice. In addition, ex vivo pretreatment of white adipose tissues from mice on a HFD with amlexanox enhanced glycerol release (FIG. 5B). This effect was more pronounced in the inguinal fat depot, where amlexanox pretreatment increased phosphorylation of HSL, perilipin, and other proteins recognized by the PKA substrate motif antibody in response to CL-316,243 treatment compared to vehicle-pretreated tissues (FIG. 5C). Amlexanox also concurrently increased the phosphorylation of TBK1 at Ser172 due to the relief of feedback inhibition, as previously reported with other inhibitors (Clark et al., 2009; Reilly et al., 2013).

To examine whether inhibition of TBK1 and IKKε with amlexanox reverses resistance to catecholamineinduced lipolysis in vivo by increasing stimulation of cAMP production, cAMP levels we measured in epididymal adipose tissue from mice on HFD after CL-316,243 IP injection. Levels of cAMP were increased after CL-316,243 IP injection in mice on HFD pretreated with amlexanox (FIG. 5D). Consistent with this, HSL phosphorylation was also increased after CL-316,243 IP injection of HFD-fed mice pretreated with amlexanox (FIG. 5E).

To examine whether inhibition of catecholamine resistance in obese adipose tissue by targeting noncanonical IKKs with amlexanox can lead to increase energy expenditure in diet-induced obese mice, oxygen consumption rates were measured of vehicle or amlexanox-treated HFD-fed mice after a single injection of CL-316,243 in metabolic cages. The effect of CL-316,243 to increase energy expenditure was more pronounced in amlexanoxtreated HFD-fed mice, as compared to vehicle-treated HFD-fed mice (FIG. 5F). These data indicate that targeting the noncanonical IKKs with the selective inhibitor amlexanox ameliorated catecholamine resistance in obese adipose tissue.

1. A method of treating a subject having a condition associated with obesity, insulin resistance, or hepatic steatosis, comprising: administering to a subject having a condition or symptoms associated with obesity, insulin resistance, or hepatic steatosis: (i) an IKKε and/or TBK1 inhibitor, and (ii) a beta adrenergic agonist or sympathetic nervous system activator, such that said condition or symptoms are reduced or eliminated.

2. The method of claim 1, wherein the administering causes a reduction of body fat in the subject.

3. The method of claim 1, wherein the subject has or is at risk of experiencing obesity, diabetes, or insulin resistance.

4. The method of claim 3, wherein the diabetes is type II diabetes.

5. The method of claim 1, wherein the treatment results in increased glucose metabolism, reduction in body fat, lack of increase in body fat, increased insulin receptor signaling, decreased level of insulin receptor phosphorylation, reduction in or prevention of chronic inflammation in the liver, reduction in or prevention of chronic inflammation in adipose tissue, reduction in or prevention of hepatic steatosis, promotion of metabolic energy expenditure, reduction in circulating free fatty acids, or reduction in cholesterol.

6. The method of claim 1, wherein the subject has hepatic steatosis (fatty liver disease).

7. The method of claim 6, wherein the subject also has steatohepatitis.

8. The method of claim 1, wherein the subject is overweight or obese.

9. The method of claim 1, wherein the subject is human.

10. The method of claim 1, further comprising a step comprising testing the subject for a disease or condition selected from the group consisting of impaired insulin signaling, obesity, diabetes, insulin resistance, metabolic syndrome, hepatic steatosis, chronic liver inflammation, and chronic inflammation in adipose tissue.

11. The method of claim 10, further comprising the step of assessing the effectiveness of treatment based upon said testing.

12. The method of claim 11, further comprising adjusting the treatment based on said assessing.

13. The method of claim 12, wherein adjusting the treatment comprises one or more of altering the dose of IKKε/TBK1 inhibitor, switching to a different IKKε/TBK1 inhibitor, altering the dose of beta adrenergic agonist or sympathetic nervous system activator, switching to a different beta adrenergic agonist or sympathetic nervous system activator, adding additional treatment.

14. The method of claim 1, wherein the IKKε and/or TBK1 inhibitor, and the beta adrenergic agonist or sympathetic nervous system activator are co-formulated in a single pharmaceutical composition.

15. The method of claim 1, wherein the IKKε and/or TBK1 inhibitor, and the beta adrenergic agonist or sympathetic nervous system activator are separate pharmaceutical composition and are co-administered.

16. A pharmaceutical composition comprising: (i) an IKKε and/or TBK1 inhibitor, and (ii) a beta adrenergic agonist or sympathetic nervous system activator.

17. The pharmaceutical composition of claim 16, wherein the IKKε and/or TBK1 inhibitor comprises a small molecule.

18. The pharmaceutical composition of claim 17, wherein the IKKε and/or TBK1 inhibitor comprises the structure of Formula I: 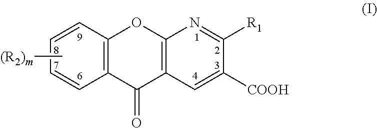
wherein R₁ is a hydrogen, alkyl, phenyl, carboxyl, hydroxyl, alkoxy, or amino group, which may be unsubstituted or substituted by one alkyl; m is 0, 1 or 2; and R₂ is alkyl, alkoxy, halogen, nitro, hydroxy, carboxyl, butadienylene (—CH═CH—CH═CH—), which forms a benzene ring with any adjacent carbon atoms or amino group, which may be unsubstituted or substituted by at least one alkyl, and their physiologically acceptable salts.

19. The pharmaceutical composition of claim 18, wherein the IKKε and/or TBK1 inhibitor comprises amlexanox.

20. The pharmaceutical composition of claim 16, wherein the beta adrenergic agonist or sympathetic nervous system activator comprises a small molecule.

21. The pharmaceutical composition of claim 20, wherein the beta adrenergic agonist or sympathetic nervous system activator comprises a β2 adrenergic receptor agonist.

22. The pharmaceutical composition of claim 20, wherein the small molecule is phentermine.

23. A kit or system comprising: (i) an IKKε and/or TBK1 inhibitor, and (ii) a beta adrenergic agonist or sympathetic nervous system activator.
